(19) United States
(12) Patent Application Publication
(10) Pub. No.: US 2023/0190241 A1
(43) Pub. Date: Jun. 22, 2023

(54) PROBE WITH COOLING CHAMBER

(71) Applicant: SUPERSONIC IMAGINE, AIX-EN PROVENCE (FR)

(72) Inventors: Matthieu VALENTIN, Marseille (FR); Etienne ROUSSEAU, Aix En Provence (FR); Emilien ROUCHON, Tarascon (FR)

(21) Appl. No.: 17/925,275

(22) PCT Filed: May 12, 2021
(86) PCT No.: PCT/FR2021/050838

§ 371 Date: Nov. 14, 2022

(30) Foreign Application Priority Data
May 15, 2020 (FR) 20 04859

(51) Int. Cl. A61B 8/00; G01S 7/52

(52) U.S. Cl. CPC A61B 8/546; G01S 7/5205; G01S 7/52079

(57) ABSTRACT

A probe, in particular for ultrasound, having a cooling chamber which is fluidtight and at least partially filled with a dielectric heat-transfer fluid. An interface unit associated with an emitting and/or receiving element of the probe is located at least partially inside or in contact with the cooling chamber. The probe includes a dry chamber separated from the cooling chamber by a fluidtight wall, and a porous heat sink arranged at least partially inside the cooling chamber.

### TECHNICAL FIELD

This invention relates to probes, in particular those for ultrasound.

### PRIOR ART

More particularly, the invention relates to a probe that can be part of an ultrasound imaging system.

Ultrasound imaging works by emitting ultrasound waves into a medium and recording the ultrasound waves reflected by the medium.

To this end, a probe is used to position the wave-emitting/receiving elements at the element to be imaged, typically an organ in the body of a living being. The waves are created by actuators, for example actuators of the piezoelectric or CMUT type which, during operation, produce heat in an induced manner. This heat may be a function of the power of the waves emitted. Patent application US 2009/234230 describes an example of a probe that emits at high frequency.

This induced heat may sometimes reach a temperature which can be uncomfortable for the patient and/or practitioner, and/or does not comply with standards in effect for medical devices. In addition, an increase in temperature in the probe can temporarily or permanently reduce its capabilities. Mechanisms are sometimes provided to prevent the probe from being damaged by heat: for example, the probe’s power may be curbed when a certain temperature is reached, in order to protect its internal components. For example, the threshold temperature may be between 50° C. and 80° C. The probes may also be programmed to shut off when this threshold temperature is reached, in order to allow the probe to cool down. In addition, such heating can reduce the pace of the probe’s ultrasound emission sequences for creating images. It is therefore necessary to provide a probe that overcomes these problems, while allowing a potentially intensive use and while maintaining optimal production and operating costs.

### DISCLOSURE OF THE INVENTION

The object of this invention is to improve probes of the type mentioned below, in order to avoid excessive heating of the probe or even damage to it.

The invention thus relates to a probe, in particular for ultrasound, which comprises:
a casing defining an interior and an exterior of the probe,
one or more emitting and/or receiving elements for acoustic waves,
an interface unit associated with the emitting and/or receiving element, the interface unit being located within the interior of the casing, the probe being characterized in that it comprises:
   a cooling chamber that is fluidtight and formed within the interior of the casing, the cooling chamber being at least partially filled with a dielectric heat transfer fluid, the interface unit being at least partially located within or in contact with the cooling chamber so as to be at least partially in contact with the heat transfer fluid,
   a dry chamber formed within the interior of the casing of the probe, the dry chamber being separated from the cooling chamber by a fluidtight wall, and
   a porous heat sink arranged at least partially within the cooling chamber.

Due to the presence of at least one cooling chamber, the temperature of the probe during operation can be effectively reduced while not requiring the probe to be shut off in order to achieve this.

During operation, the plurality of emitting and/or receiving elements for acoustic waves produce heat, and the heat transfer fluid shifts this heat, emitted at the end of the probe comprising the plurality of emitting and/or receiving elements, towards a second end of the probe, the second end being distal to the plurality of emitting and/or receiving elements for acoustic waves.

The porous heat sink advantageously makes it possible to increase the heat exchanges, in particular between these two ends of the probe.

In various embodiments of the probe according to the disclosure, one or more of the following arrangements may possibly be used:
the fluidtight wall is at least partially composed of an intermediate board.
the intermediate board is composed of a multilayer printed circuit and a plurality of filled vias traversing some or all of these layers.
the porous heat sink is formed of open-cell pores.
the heat transfer fluid is phase changing.
the heat transfer fluid is a single-phase fluid, in the liquid state.
the heat transfer fluid is a hydrofluoroether.
the fluid has a transition temperature which depends on the temperature resulting from the heat produced by the emitting and/or receiving element for acoustic waves during operation, such that when the probe is used, the heat transfer fluid can be in both the gas phase and the liquid phase.
the transition temperature of the heat transfer liquid is between a temperature of 30 and 90 degrees Celsius.
the interface unit traverses the fluidtight wall and is arranged partly in the dry chamber and partly in the cooling chamber.
a part of the interface unit is composed of at least one printed circuit located in the cooling chamber, said at least one printed circuit being mounted on both sides of the porous heat sink.
another part of the interface unit is composed of an intermediate board which traverses the fluidtight wall, the intermediate board being associated with said at least one printed circuit (228, 328, 428).
at least part of the interface unit comprises a flexible or semi-rigid material.
the interface unit is connected to the exterior of the probe by a plurality of cables, the plurality of cables being located in the dry chamber.
the porous heat sink includes pores having a diameter within an interval of ]0; 10] mm.
the porous heat sink includes pores of variable size.
the pores are of increasing size along a direction oriented towards a center of the cooling chamber.
the pores are of decreasing size along a direction oriented towards a center of the cooling chamber.
the porous heat sink comprises several porous layers.
each layer of the porous heat sink has a different porosity size than the other layers.
the porous heat sink is in contact with the interface unit.
the porous heat sink is arranged between said one or more emitting and/or receiving elements and the interface unit.
the porous heat sink is obtained by powder sintering.
the porous heat sink is a first heat sink, the probe comprising a second heat sink arranged in the dry chamber or on a face of the fluidtight wall in contact with the dry chamber.
the second heat sink comprises a porous material.
the porous heat sink has a shape complementary to the walls of the cooling chamber and/or at least partially fills the volume of the cooling chamber.
the porous heat sink is in contact with both a rear face of said one or more emitting and/or receiving elements and the fluidtight wall.
the probe further comprises a pressure sensor and/or an impact sensor and/or a temperature sensor within the interior or associated with the probe.
said one or more emitting and/or receiving elements for acoustic waves are arranged at a first end of the probe, the cooling chamber is arranged towards the first end of the probe, and the dry chamber is arranged towards a second end of the probe, the second end being opposite to the first end.

### BRIEF DESCRIPTION OF DRAWINGS

Other features and advantages of the invention will become apparent from the following description of some of its embodiments, given as non-limiting examples, with reference to the attached drawings.

In the drawings:

FIG. 1a is a front cross-section of a probe according to a first embodiment;

FIG. 1b is a side cross-section of the probe of FIG. 1a;

FIG. 2 is a front cross-section of a probe according to a second embodiment;

FIG. 3 is a front cross-section of a probe according to a third embodiment;

FIG. 4 is a front cross-section of a probe according to a fourth embodiment;

FIG. 5a is a front cross-section of a probe according to a fifth embodiment; and

FIG. 5b is a side cross-section of the probe of FIG. 5a.

In the various figures, unless otherwise stipulated, the same numerical references designate identical or similar elements.

### DETAILED DESCRIPTION

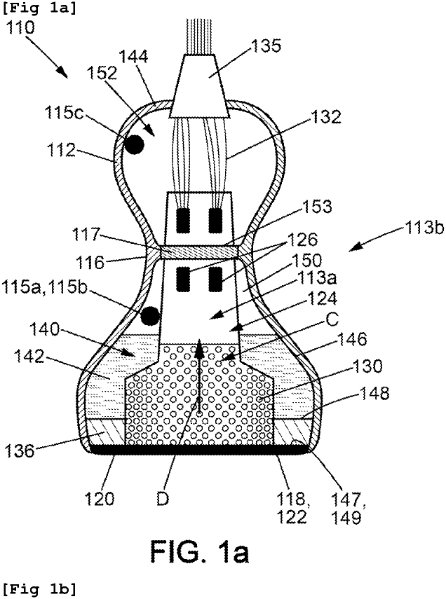

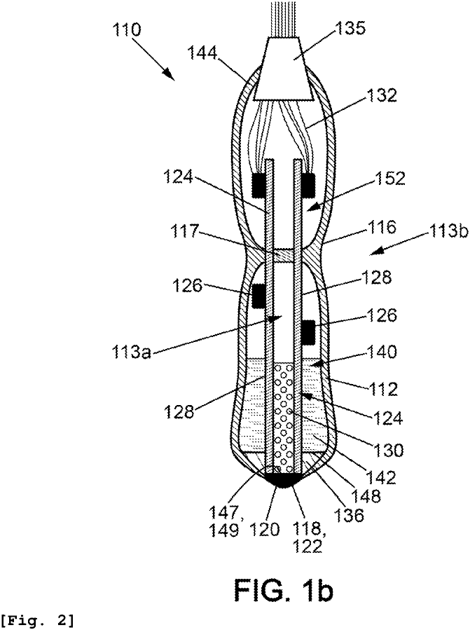

With reference to FIGS. 1a and 1b, a probe 110 according to a first embodiment is described, in particular for acoustic waves, for example ultrasound. In one embodiment, the probe 110 is part of an ultrasound imaging system. This system may be used for example in a medical context for the visualization of internal or external organs and/or tissues, for example of the body of a human or animal. For this purpose, the probe 110 allows emitting and/or receiving ultrasonic waves.

The probe 110 includes a casing 112 which is a shell containing the various components of the probe 110. The casing 112 separates an interior 113a from an exterior 113b of the probe 110. The casing 112 is for example generally rigid, to facilitate manipulation of the probe 110. The casing 112 includes a generally ergonomic gripping portion 116, by means of which a user manipulates the probe, generally with one hand.

The casing 112 is also composed of an emitting and/or receiving surface 118 which is suitable for contact with a tissue, for example the skin, or any wave-transmitting medium, for example such as tissue, an object, an ultrasound gel. The emitting and/or receiving surface 118 is illustrated in the figures as being generally flat, or even with a slight curvature, planar or in 2D. However, the emitting and/or receiving surface 118 may have various 3D shapes, possibly with a pronounced curvature.

The casing 112 may be manufactured in part from one or more electrically insulating materials such as plastic, for example of the ABS type. According to one embodiment, the casing 112 is made of metal. The metal casing 112 can make it possible to increase heat dissipation, but can also serve as electromagnetic shielding by forming a Faraday cage. In one variant, the casing 112 may be composed of several assembled parts. For example, the casing 112 (without the emitting and/or receiving surface 118) could consist of a shell formed of a single piece of plastic leaving an opening suitable for receiving the emitting and/or receiving surface 118 therein, thus connected and closing the casing 112. The casing 112 may be rigid or flexible, in whole or in part. According to one example, the emitting and/or receiving surface 118 is composed of one or more flexible polymer(s). The casing 112 may be made by molding or by 3D printing.

The casing 112 may be fabricated to be fluidtight to liquids and gases.

The probe 110 includes, positioned wholly or partly within the interior 113a of the casing 112, a plurality of emitting and/or receiving elements 120 for acoustic waves arranged at a first end 122 of the probe 110. In one variant, it is possible that the probe 110 comprises a single emitting and/or receiving element 120; however, for simplification, this term will be used interchangeably with the plural in the present description. The first end 122 is an end of the probe 110 which comprises the emitting and/or receiving surface 118 of the casing 112. According to one embodiment, the plurality of emitting and/or receiving elements 120 comprises a plurality of piezoelectric elements (a single piezoelectric element in the variant presented above). The plurality of emitting and/or receiving elements 120 may be arranged so as to form an emission row or edge, or an emission surface, for example forming a rectangle.

The plurality of emitting and/or receiving elements 120 is associated with an interface unit 124 which is located within the interior 113a of the probe 110 and in contact with a rear face 147 of the plurality of emitting and/or receiving elements 120, so as to transmit emission signals and/or to receive reception signals or reception data and to communicate with a control unit of the ultrasound imaging system. In the embodiment of FIGS. 1a and 1b, the interface unit 124 includes two printed circuit boards 128 (or printed circuits) facing each other, each having one or a plurality of electronic components 126, active and/or passive, depending on the embodiments. In one embodiment, the interface unit 124 includes one or more than two printed circuit boards 128. The printed circuit boards 128 may be rigid, semi-rigid, or fully flexible. The architecture of the flexible printed circuit boards 128 may advantageously allow better dispersion of the heat transfer fluid within the heat sink, as will be understood from the description below. The interface unit 124 may comprise more or fewer active electronic components, one or more pulsers, an antenna, and/or one or more microprocessor(s). According to one embodiment, the interface unit 124 is composed only of passive elements, of the connector type, and constitutes a relay for signals or information with the control unit of the imaging system. The interface unit is then a structure which allows grouping electrical connections to the emitting and/or receiving elements 120, for example via one or more connectors and/or multiplexers.

According to another embodiment, the interface unit 124 comprises active elements and participates at least partially in the generation of emission signals intended for the plurality of emitting and/or receiving elements 120. In one variant, part of the control unit of the imaging system can be relocated to the interface unit 124. According to one embodiment, the interface unit 124 is directly in contact with the plurality of emitting and/or receiving elements 120 along their entire length.

The interface unit 124 communicates with an ultrasound imaging system (not shown), in one embodiment, via a plurality of cables 132. According to one embodiment, the cables 132 are coaxial cables. According to another embodiment, the cables 132 are non-coaxial cables, optical fibers, or any other technical means which allow transferring/exchanging signals and/or data between two entities. According to one embodiment, the cables 132 are absent: the interface unit 124 communicates with the ultrasound imaging system wirelessly, for example by means of an antenna placed in the probe or on the probe.

The cables 132 traverse the casing 112 via a cable sleeve 135 at a second end 144 of the probe 110. The cable sleeve 135, according to one embodiment, may be fluidtight to liquids and gases. The second end 144 is opposite to the first end 122, and in this variant is distal to the plurality of emitting and/or receiving elements 120 for acoustic waves. In some variants, the cable sleeve 135 which the cables 132 pass through may be located at another location of the casing 112. The cables 132 are connected to the interface unit 124 by one- or two-dimensional connectors.

A wave-blocking element 136 may be located on a portion of the rear face 147 of the plurality of emitting and/or receiving elements 120 where the interface unit 124 is not in contact with the plurality of emitting and/or receiving elements 120. The wave-blocking element 136 provides at least partial acoustic damping between the plurality of emitting and/or receiving elements 120 and a portion of the interior 113a of the probe 110. Indeed, when the plurality of emitting and/or receiving elements 120 is in operation, the created acoustic waves can propagate towards the interior 113a of the probe 110 and bounce off the various elements located inside the probe, and thus create unwanted acoustic noise. The purpose of the wave-blocking element 136 is to block the vast majority of these waves propagating towards the interior 113a of the probe 110, by returning them or else absorbing them. The wave-blocking element 136 also makes it possible to shorten the duration of the emission pulse. The wave-blocking element 136 is for example an elastomer, a flexible resin, or a composite foam. The blocking element may comprise metal particles in order to match the impedance. The wave-blocking element 136 may additionally or alternatively be chosen to be a heat-conducting element.

The probe 110 includes a cooling chamber 140 for cooling the heating of the probe 110 induced by the operation of the plurality of emitting and/or receiving elements 120. The cooling chamber 140 is formed within the interior 113a of the casing 112 towards the first end 122 of the probe 110.

The cooling chamber 140 is fluidtight (to liquids and gases) and comprises a dielectric heat transfer fluid 142 which partially or entirely fills it. The interface unit 124 is at least partially in contact with the heat transfer fluid 142. According to one embodiment, the heat transfer fluid 142 is single-phase, preferably liquid. A liquid single-phase fluid may be preferred over a two-phase fluid or a gaseous single-phase fluid for reasons concerning possible leaks, depending on the construction of the casing 112. According to one embodiment, the fluid has a relatively high viscosity, for example a dynamic viscosity at 40° C. that is greater than or equal to 7.7 mm²/s.

An example of a single-phase dielectric heat transfer fluid is MIVOLT CL200 and MIVOLT CL300. The density of MIVOLT CL300 at 20° C. is 0.97 g/cc, calculated according to ISO 3675. The density of MIVOLT CL200 at 20° C. is 0.92 g/cc, calculated according to ISO 3675. These fluids are not traditionally used in the medical field, but constitute a heat transfer fluid of interest for the probe 110.

According to one embodiment, the heat transfer fluid 142 is biocompatible. This choice of fluid may be of interest for applications involving the use of endoscopic probes, for example.

According to one embodiment, the heat transfer fluid 142 is a two-phase fluid such that, for the operating temperatures of the probe 110, it is potentially simultaneously in the liquid phase and in the gaseous phase. In addition, the heat transfer fluid 142 may be a heat transfer fluid 142 of a nature compatible with the elements of the probe 110 with which it is in contact, so as not to damage these elements. Otherwise it is insulated, for example in a pouch made of an insulating material such as polytetrafluoroethylene (PTFE), nylon, polypropylene, stainless steel, aluminum, or copper. The heat transfer fluid 142 is non-solid and essentially non-gaseous within a normal operating range during use (i.e. in the liquid phase for the most part). For example, the nominal operating temperature of the probe can vary between 5° C. and 35° C. (°C being the unit of degrees Celsius). The heat transfer fluid 142 used may alternatively have a boiling (or transition) temperature close to the local temperature inside the probe 110 when the plurality of emitting and/or receiving elements 120 are in operation. For example, the heat transfer fluid 142 may be selected to have a boiling temperature close to a local temperature of the wave-blocking element 136 or of the interface unit 124, the emitting and/or receiving elements being in operation. According to one embodiment, the transition temperature of the heat transfer fluid 142 is between ambient temperature (between 15° C. and 25° C.) and an extreme temperature (between 60° C. and 90° C., °C being the unit of degrees Celsius). The transition temperature of the heat transfer liquid 142 may be between a temperature of 30 and 90 degrees Celsius. The transition temperature is for example between 30° C. and 40° C., and optionally 34 degrees Celsius. According to one embodiment, the heat transfer fluid 142 has a viscosity approximately 5 times lower than that of water, for example a viscosity of between 0.32 cSt to 0.8 cSt. The heat transfer fluid 142 is for example water, oil, an alcohol, an ether, a fluorocarbon, a hydrofluoroether (HFE) for example of the 3M Novec 7000™ type, or a perfluorocarbon (PFC), or any mixture of these compounds. Novec 7000™ has a lower viscosity than MIVOLT CL300 and MIVOLT CL200. The density of Novec 7000™ is 1.40 g/mL at 25° C. Cooling by Novec 7000™ then primarily occurs by convection due to the low boiling temperature as well as its low viscosity.

Due to the presence of the heat transfer fluid 142, passive cooling of the interface unit 124 is achieved. This makes it possible to do without a complex active cooling system, for example a system using a pump with a fluid passing through cables or hoses connected to the probe 110.

In the example of FIGS. 1a and 1b, the interface unit 124 is located partially in the cooling chamber 140. According to other embodiments, of which some will be described and illustrated below, the interface unit 124 is completely contained within the cooling chamber 140, or the interface unit 124 is partially in contact with the cooling chamber 140 for example by a wall.

In the embodiment of FIGS. 1a and 1b, the cooling chamber 140 is coincident with the interior 113a of the casing 112. The cooling chamber 140 is therefore defined by an inner wall 146 of the casing 112 and by a rear face 148 of the wave-blocking element 136 (a front face 149 of the wave-blocking element 136 being in contact with the plurality of emitting and/or receiving elements 120). The wave-blocking element 136 is fluidtight to liquids and gases, such that the heat transfer fluid 142 does not enter the first end 122 of the probe 110 containing the plurality of emitting and/or receiving elements 120. According to other alternatives, the cooling chamber 140 may not be in direct contact with the rear face 148 of the wave-blocking element 136. An intermediate sealing element may for example be there.

The cooling chamber 140 is, according to one embodiment, partially filled with the heat transfer fluid 142, such that another part of the cooling chamber 140 is filled with a gas 150. The gas 150 is a compressible gas, for example air. According to one embodiment, the cooling chamber 140 is filled with heat transfer fluid 142 at 45% by volume and gas 150 at 55% by volume. According to one embodiment, the cooling chamber 140 is mainly filled with heat transfer fluid 142 and the remainder of the volume of the cooling chamber 140 is filled with gas 150.

According to one embodiment, the cooling chamber 140 is filled with heat transfer fluid 142 to at least 5% by volume and conversely with gas 150 up to a maximum of 95% by volume. According to one embodiment, the cooling chamber 140 is filled with heat transfer fluid 142 to at least 10% by volume and conversely with gas 150 up to a maximum of 90% by volume. The filling percentage may depend on the nature of the heat transfer fluid 142 and/or of the gas 150 and/or of the cooling chamber 140 and/or of any element of the probe. This filling percentage can be optimized by tests. According to another embodiment, the cooling chamber 140 is completely filled with the heat transfer liquid 142. This may be the case when the heat transfer liquid 142 is not phase changing. A portion 114 of the cooling chamber 140 (which may or may not be a portion of the casing 112) may be flexible, so as to serve as a membrane compensating for a variation in volume, for example due to the phase change of the heat transfer fluid 142. In certain variants, it is possible to eliminate the compensating membrane 114, in particular when the walls of the cooling chamber (the casing 112 or other element depending on the embodiments) have sufficient flexibility.

During operation, the plurality of emitting and/or receiving elements for acoustic waves 120 produce heat, and the heat transfer fluid 142 transfers, by convection and/or convection, an amount of this heat towards the second end 144 which is a naturally colder area than the area of the plurality of emitting and/or receiving elements 120, which thus has the effect of cooling the probe 110. It is possible to obtain cooling of the probe 110 by means of various characteristics of the heat transfer fluid 142. The cooling may take place by convection/conduction in the liquid phase of the heat transfer fluid 142 and by phase change when the local temperature reaches the phase change temperature and part of the heat transfer fluid 142 turns into gas.

The volume portions of the heat transfer fluid 142 that are heated during operation of the plurality of emitting and/or receiving elements 120 move towards an area of the liquid portions of the heat transfer fluid 142 which is colder, typically towards the area furthest away of the plurality of emitting and/or receiving elements 120. During this movement, the cooler volume portions are pushed towards the plurality of emitting and/or receiving elements 120. They are then in turn “heated” by the heat released by the plurality of emitting and/or receiving elements 120 during operation, and a movement, which may be continuous, of convection and/or conduction of the heating and cooling of the volume portions of the heat transfer fluid 142 is thus produced.

When the wave-blocking element 136 reaches the phase transition temperature, the heat transfer fluid 142 in the liquid phase begins to boil. The gas thus generated moves towards the second end 144 which is naturally colder than the area of the probe 110 that is close to the emitting and/or receiving elements. Contact with this colder area condenses the gas, which is transformed into droplets of heat transfer fluid 142 and which reaches the first end 122 by liquid flow, thus joining the heat transfer fluid 142 which has not yet evaporated. Part of the heat is also optionally extracted or dissipated through the walls of the casing 112.

In one embodiment, the interface unit 124 is sealed off by adding a coating and/or is made resistant to warm and humid conditions before insertion into the cooling chamber 140, which makes it possible, for example, to avoid contact with the fluids to be found therein. An epoxy resin may thus be applied as a protective coating all around the interface unit 124, in order to isolate it from the heat transfer fluid 142.

The probe 110 further comprises a dry chamber 152 within the interior 113a of the casing 112. The dry chamber 152 is separated from the cooling chamber 140 by a fluidtight wall 153 impermeable to liquids and gases.

The cooling chamber 140 is generally located towards the first end 122 of the probe 110, and the dry chamber 152 is towards the second end 144 of the probe 110. Since the cooling chamber 140 contains fluid, by positioning the cooling chamber 140 generally towards the first end 122, the center of gravity is moved towards the first end 122, which advantageously makes manipulation of the probe 110 easier and more comfortable.

According to the embodiment of FIGS. 1a and 1b, the dry chamber 152 is formed by the inner wall 146 of the casing 112. According to another embodiment, the dry chamber 152 is only partially formed by the inner wall 146 of the casing 112, or is separate from the inner wall 146 of the casing 112. In the embodiment of FIGS. 1a and 1b, the dry chamber 152 and the cooling chamber 140 are shown as together filling the interior 113a of the probe 110. Alternatively, the dry chamber 152 and/or the cooling chamber 140 may be located within the interior 113a of the casing 112 without being delimited by the casing 112, and/or the probe 110 contains other chambers besides the cooling chamber 140 and dry chamber 152.

The dry chamber 152 is traversed by the cables 132 which connect the interface unit 124 to the exterior 113b of the probe 110. According to one embodiment, the cables 132 of the probe 110 are only located within the dry chamber 152. The dry chamber 152 comprises a gas according to one embodiment, for example air.

The fluidtight wall 153, according to the embodiment of FIGS. 1a and 1b, may be formed of a part 117 which extends between the two printed circuit boards 128, and which is connected to them in a fluidtight manner (i.e. fluidtight to liquids and/or gases). The part 117 can make it possible to keep the printed circuit boards 128 parallel to each other. The interface unit 124 may be connected in a fluidtight or sealed manner to the inner wall 146 of the casing 112. This fluidtight connection at the part 117 and at the casing 112 may be achieved by an epoxy resin (which could also cover the interface unit 124) and/or an elastomer seal chemically compatible with the heat transfer fluid 142 so that the heat transfer fluid 142 does not damage the elastomer seal. The part 117 may be made of one or more material (s) enabling heat exchange, comprising for example copper and/or aluminum.

According to one embodiment, the fluidtight wall 153 may have a filling device which allows filling the cooling chamber 140 with heat transfer fluid during manufacture, once the fluidtight wall 153 is sealed to the casing 112 or during subsequent maintenance phases. This filling device is for example a valve, an elastic membrane, an orifice closed by a stopper, or a ball set in an elastomer hose.

A heat sink 130 may be placed in the cooling chamber 140 in order to improve heat exchanges locally. The heat sink 130 according to this disclosure may comprise at least one portion composed of a porous material. A porous material is a continuous medium with cells corresponding to cavities or pores able to contain one or more fluids or gases. These pores may have constant or variable sizes. Advantageously, these cavities or pores are open or are through-holes in order to form channels capable of allowing the flow of fluid(s) in order to improve heat exchange. For example, the porous material may be a solid of detailed geometry containing pores or cells of small size and which may contain one or more fluids (liquid and/or gas). Examples of porous materials are sintered materials or metal foams. The heat sink 130 may be a porous prismatic metal piece or porous fins. The presence of porosity in the heat sink 130 coupled with heat dissipation by conduction can be particularly effective with a heat transfer fluid 142 such as MIVOLT CL200.

In one embodiment, the printed circuit boards 128 are assembled one on each side of the porous heat sink 130. In the embodiment of FIGS. 1a and 1b, the heat sink 130 is sandwiched between the two printed circuit boards 128. According to one embodiment, the heat sink 130 extends from the rear face 147 of the plurality of emitting and/or receiving elements 120 to an intermediate height relative to the printed circuit boards 128, as shown in FIGS. 1a and 1b. According to another embodiment, the heat sink 130 extends from the rear face 147 of the plurality of emitting and/or receiving elements 120 to the fluidtight wall 153, thus covering the entire space between the two printed circuit boards 128.

The heat sink 130 may, according to one embodiment and as illustrated in FIGS. 1a and 1b, extend laterally beyond the printed circuit boards 128 in order to increase the heat exchanges with the heat transfer fluid in this area. It may form wings at the rear face 147 of the plurality of emitting and/or receiving elements 120. According to one embodiment, these wings may extend as far as the fluidtight wall 153.

The heat sink 130 could be located elsewhere in the cooling chamber 140, without connection to the rear face 147 of the plurality of emitting and/or receiving elements 120. It may be located, for example, at the fluidtight wall 153 of separation from the dry chamber 152.

According to one embodiment, the heat sink 130 comprises open-cell pores forming a network of cooling channels traversing the heat sink 130. According to one embodiment, the heat sink 130 is a porous medium made of a foam, such as a metal foam.

According to one embodiment, the pores are sized to allow flow of the liquid phase of the heat transfer fluid 142, thus facilitating the heat exchange between the heat transfer fluid 142 and the interface unit 124.

The heat sink 130 may be partially or fully porous. For example, the heat sink 130 may have a non-porous portion at the location where it is in contact with another element of the probe 110 (for example the rear face 147 of the plurality of emitting and/or receiving elements 120 and/or the inner faces of the printed circuit boards 128).

The heat sink 130 may have pores of generally uniform size, or of varying sizes. The pores may be of random sizes or follow an increasing or decreasing evolution or an alternation between the two. For example, the pores may be of increasing size along a direction going in a direction D oriented towards a center C of the cooling chamber 140. In another example, the pores are of decreasing size along the direction D oriented towards the center C of the cooling chamber 140. A low porosity or no porosity near the wave-blocking element 136 makes it possible to better transmit heat in the heat sink 130. At a greater distance from the wave-blocking element 136, a greater porosity can allow circulation of the heat transfer fluid 142 and improve the heat exchange between the heat sink 130 and the heat transfer fluid 142 all along the heat sink 130. A porosity that becomes smaller along direction D as one approaches the fluidtight wall 153 and/or the inner walls 146, starting from the center C of the cooling chamber 140, can condense the fluid 142 gasified by heat from the interface unit 124. Thus, along direction D, the porosity can increase starting from the wave-blocking element 136 and approaching the center C, then decrease starting from the center C and approaching the walls 146 and/or the fluidtight wall 153.

In one embodiment, the heat sink 130 includes pores having a width within an interval of ]0; 10] mm (for a millimeter standard unit of measurement). In one embodiment, the heat sink 130 includes pores having a width within an interval of ]0; 6] mm.

The heat sink 130 may be made as a single piece or may be composed for example of several layers, of the same materials or of different materials. Each layer (or some layers) may have a pore size (or porosity) specific to it. In another embodiment, each layer (or some layers) may have the same pore size.

The heat sink 130 may be made of several types of heat sinks: for example it may comprise fins with no porosity in addition to the porous structure, or maybe a porous drain (generally prismatic piece of metal) to which fins are added.

The heat sink 130 may be made of one or more heat-conducting materials, such as a ceramic and/or a metal. According to one embodiment, the heat sink 130 is made of copper or aluminum. It may be made by compression or sintering of beads or powder. The size of the beads or powder particles used in the compression or sintering can determine the resulting size of the cavities or pores of the heat sink 130.

According to one embodiment, the probe 110 further comprises a pressure sensor 115a and/or one (or more) temperature sensor 115b and/or an impact sensor 115c which may (may all) be located within the cooling chamber 140 or elsewhere within the interior 113a of the probe 110.

The pressure sensor 115a may for example be located on one of the printed circuits 128, and the temperature sensor 115b on the wave-blocking element 136. The pressure sensor 115a and/or the temperature sensor 115b and/or the impact sensor 115c may be functionally connected to at least one of the printed circuits 128 of the interface board 124 in order to relay information to the control unit of the ultrasound system. Optionally, the pressure 115a, temperature 115b, and impact 115c sensors are for example directly implemented on a printed circuit 128 of the interface unit 124. In one embodiment, each sensor may be “independent”, in the sense that it is, for example, energy self-sufficient and sends the measured values to the exterior of the probe independently.

The pressure sensor 115a may be used to detect possible leaks of gas 150 or liquid 142, or system malfunctions. The temperature sensor 115b allows, in one embodiment, detecting if the probe 110 has reached a temperature above a threshold which causes a contact temperature of the probe with the patient’s skin that is above 43° C. +/- 3° C. according to the IEC 60601-1 standard.

One or more of the sensors allow detecting an operating anomaly of the probe (for example liquid or gas leak and/or excessive temperature), and an alarm may be activated, the probe 110 then being able to be restricted in its operation or even shut off either directly by a circuit of the interface unit 124, or indirectly by the control unit of the ultrasound system. The impact sensor 115c is, for example, an accelerometer, and may be used to cut off the electrical functions of the probe 110 in the event of an impact. According to one embodiment, the probe 110 comprises at least one pressure sensor 115a and one temperature sensor 115b which, by combining their information, can be used to detect any leaks of heat transfer fluid 142 during operation of the probe 110.

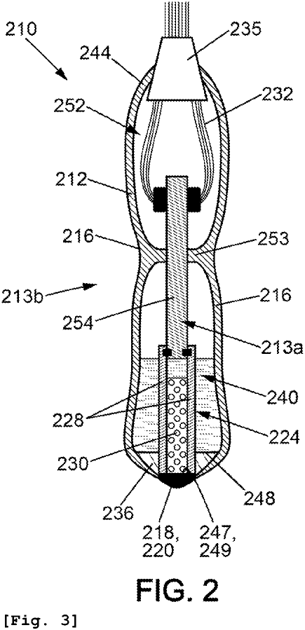

According to another embodiment and as illustrated in FIG. 2, a probe 210 is similar to the probe 110 of the first embodiment and its alternatives. The same numerical references as for probe 110 will be adopted for probe 210 except that they will be in the two hundreds. For example, the probe of the first embodiment is probe 110 and that of the second embodiment is probe 210.

For brevity, the parts common to probes 110 and 210 will not be explained again. Probe 210 has similar features to probe 110 and its alternatives, except that the two printed circuit boards 228 do not traverse the fluidtight wall 253. Instead, an intermediate board 254 traverses the fluidtight wall 253. The intermediate board 254 is connected to one or both printed circuit boards 228, and to the cables 232. By means of this arrangement, the gripping portion 216 can be thinner and therefore advantageously more ergonomic and/or light. This arrangement can also facilitate assembly of the various components of the probe. The connections between the intermediate board 254 and the printed circuit board(s) 228 may be made by one-dimensional or two-dimensional connectors, or by soldering, such as surface soldering. According to one embodiment, the interface unit 224 comprises only one printed circuit board 228 and the intermediate board 254 is associated with this printed circuit board 228.

According to one embodiment, one or more electronic components 226 of the interface unit 224 are relocated to the intermediate board 254. According to one embodiment, the intermediate board 254 has several layers connected together by vias.

The intermediate board 254 is, in one variant, made fluidtight before insertion into the cooling chamber 240. An epoxy resin may be applied to all or part of the intermediate board 254 in order to isolate it from the heat transfer liquid 242 as explained above for printed circuit boards 128.

The fluidtight wall 253 may be formed as integral with the casing 212, as shown in FIG. 2, or may comprise a piece made fluidtight or sealed to be fluidtight with the casing 212, in a manner similar to what was presented for probe 110.

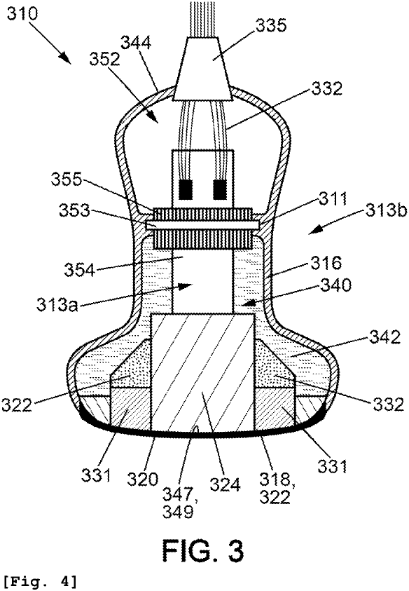

Referring to FIG. 3, a third embodiment of the probe 310 is similar to the probe 210 of the second embodiment and has the features and alternatives presented for probe 210s (and those of probe 110 via the references of probe 210 to probe 110). For brevity, the features of probes 310 in common with probes 110 and 210 will bear the same reference numbers but in the three hundreds and will not be repeated here. For example, the probe of the first embodiment is probe 110 and that of the third embodiment is probe 310.

In probe 310, the heat sink 330 comprises a non-porous part 331 in contact with the rear face 347 of the plurality of emitting and/or receiving elements 320, and a porous part 332 which extends from the non-porous part 331. The porous part 332 has the same features as those described above with reference to heat sink 130.

The probe 310 further comprises a second heat sink 354 provided in the cooling chamber 140. The second heat sink 354 is attached to the fluidtight wall 353. According to one embodiment, the fluidtight wall 353 is itself a heat sink. The fluidtight wall 353 may be made of a heat-conducting material, for example such as copper or aluminum. The second heat sink 354 increases the heat exchanges from the first end 322 of the probe 310 to the second end 344 of the probe 310. The second heat sink 354 can also allow better condensation of the vaporized heat transfer fluid 342, near the fluidtight wall 353.

The second heat sink 354 may comprise at least a portion made of porous material and have the features and alternatives discussed above for heat sink 130, for example such as non-porous fins. The second heat sink 354 could also be a combination of porous and non-porous portions, as described for heat sink 130.

The second heat sink 354 may be integrally formed with the first heat sink 530.

According to one embodiment, and as shown in FIG. 3, the probe 310 may comprise a third heat sink 355 provided in the dry chamber 352. The third heat sink 355 may be in place of or in addition to the second heat sink 354, in order to increase heat exchanges towards the second end 344 of the probe 310. The third heat sink 355 may have any configuration as those previously discussed for the first heat sink 130 and/or the second heat sink 354. Alternatively, the third heat sink 355 may be integrally formed with first heat sink 530 and/or the second heat sink 354.

According to one embodiment, the fluidtight wall 353, the second heat sink 354, and the third heat sink 355 form a part which fits into notches 311 of suitable shape and provided in the casing 312. The notches 311 allow easy assembly of the probe 310. Sealing with the casing 312 may be achieved by an epoxy resin and/or an elastomer seal chemically compatible with the heat transfer fluid 342 so that the heat transfer fluid 342 does not damage the elastomer seal, as described above. According to one embodiment, the fluidtight wall 353, the second heat sink 354, and the third heat sink 355 form a single piece.

According to one embodiment, the fluidtight wall 353 may have a filling device which allows filling the cooling chamber 340 with heat transfer fluid, once the fluidtight wall 353 is sealed to the casing 112. This filling device is for example a valve, an elastic membrane, an orifice closed by a plug, or a ball set in an elastomer hose.

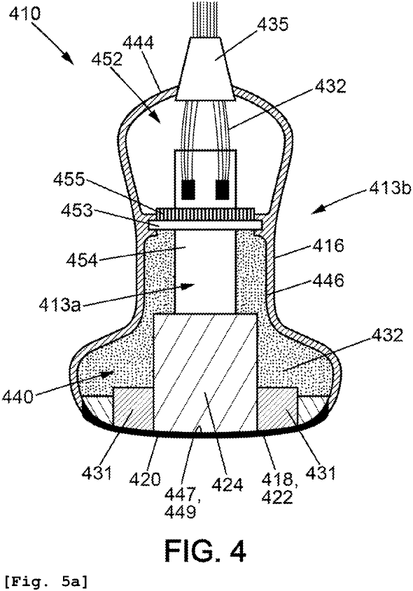

Referring to FIG. 4, a fourth embodiment of the probe 410, similar to the probe 310 of the third embodiment, has the features and alternatives shown for probe 310 (and those of probes 110 and 210 by reference). For brevity, the features of probe 410 that are in common with probes 110, 210, and 310 will bear the same reference numerals but in the four hundreds and will not be repeated here. For example, the probe of the first embodiment is probe 110 and that of the fourth embodiment is probe 410.

Probe 410 has features similar to those of probe 310 of the third embodiment, except that the porous part 432 of the heat sink 430 fills the volume V of the cooling chamber 440, such that the porous part 432 has a shape complementary to the inner wall 446 of the cooling chamber 440. The porous part 432 may be for example obtained by using a 3D printer, such as a metal powder 3D printer. The porous part 432 more or less fills the volume V of the cooling chamber 440 while having portions in contact with the inner wall 446 of the cooling chamber 440 that are of a complementary shape thereto. In the example of FIG. 4, the porous part 432 fills the volume V and therefore is coincident with the second heat sink 454 attached to the fluidtight wall 453 of the cooling chamber 440. It is conceivable, however, that in the case where the porous part 432 only partially fills the volume V while having a shape complementary to the inner wall 446 of the cooling chamber 440, the probe 410 also has the second heat sink 454 attached to the fluidtight wall 453 of the cooling chamber 440.

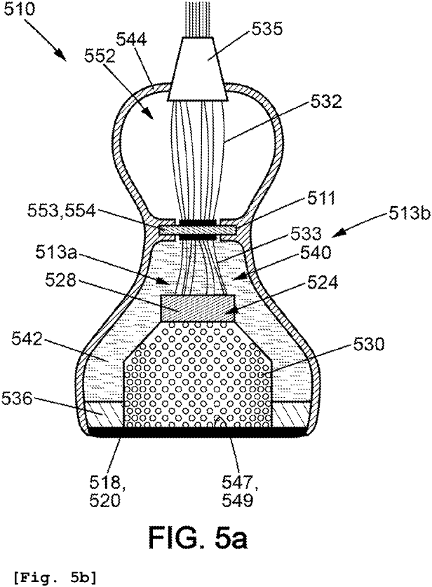

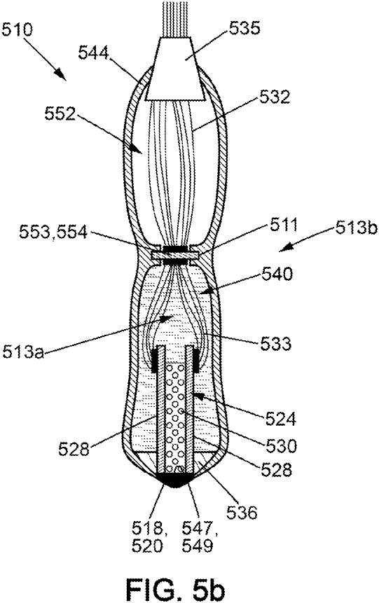

Referring to FIGS. 5a and 5b, a probe 550 according to a fifth embodiment is shown. Probe 550 has similarities to probe 110 of the first embodiment and has the alternatives shown for probe 110. For brevity, features of probe 550 that are in common with probe 110 will bear the same reference numerals but in the five hundreds and will not be repeated here.

Certain aspects of probe 550 may be combined with those presented for probes 210, 310, 410, in particular aspects relating to the heat sinks of the cooling chamber 540 and possibly the dry chamber 552. For example, the layout, size, consistency, number of heat sinks may be any of those described above with respect to probes 110, 210, 310, and/or 410, taken separately or in combination.

Probe 550 differs from probe 110 in that the fluidtight wall 553 between the cooling chamber 540 and the dry chamber 552 is an intermediate board 554. The intermediate board 554 is connected to the cables 532 in the dry chamber 552 and to the interface unit 524. Thus, the intermediate board 554 divides the cooling chamber 540 from the dry chamber 552, with a first surface of the intermediate board 554 being in the dry chamber 552, and a second surface of the intermediate board 554, opposite to the first surface, being in the cooling chamber 540.

The interface unit 524 is connected with the second surface of the intermediate board 554 located in the cooling chamber 540, by cables 533. The cables 533 are fluidtight to the heat transfer fluid 542 located in the cooling chamber 540. The cables 532 and 533 are in electrical communication by means of filled vias of the intermediate board 554. The vias are electrically conductive and they may be made of copper or tin or aluminum or any alloy of these materials. The cables 533 could be replaced by an intermediate board, or be absent if the probe is wireless as discussed above.

The intermediate board 554 is, in one embodiment, made fluidtight before being inserted into the probe 510. An epoxy resin may be applied all around the intermediate board 554 in order to isolate it from the heat transfer liquid 542.

According to one embodiment, the intermediate board 554 is composed of several layers of printed circuits. Filled vias may traverse some or all of these layers. According to one embodiment, one or more electronic components 526 of the interface unit 524 are relocated to the intermediate board 554. According to one embodiment, the intermediate board 554 has several layers connected together by vias.

According to one embodiment and as illustrated in FIGS. 5a and 5b, the interface unit 542 fits into notches 511 of suitable shape provided in the casing 512. The fluidtightness and/or the sealing with the casing 112 may be achieved by an epoxy resin and/or an elastomer seal which would be chemically compatible with the heat transfer fluid 142 so that the heat transfer fluid 142 does not damage the elastomer seal, as described above.

The fluidtight wall 553 may have heat sinks as discussed above for probe 310.

These different variants are explained for illustrative purposes only, and may be taken alone or in combination.

1. Probe in particular for ultrasound, comprising:
a casing defining an interior and an exterior of the probe,
one or more at least one of emitting and receiving elements for acoustic waves,
an interface unit associated with the one or more at least one of emitting and receiving elements, the interface unit being located within the interior of the casing, Wherein the probe comprises:
a cooling chamber that is fluidtight and formed within the interior of the casing the cooling chamber being at least partially filled with a dielectric heat transfer fluid the interface unit being at least partially located within or in contact with the cooling chamber so as to be at least partially in contact with the heat transfer fluid,
a dry chamber formed within the interior of the casing of the probe, the dry chamber being separated from the cooling chamber by a fluidtight wall, and
a heat sink arranged at least partially within the cooling chamber.

2. Probe according to claim 1, wherein the fluidtight wall is at least partially composed of an intermediate board.

3. Probe according to claim 2, wherein the intermediate board is composed of a multilayer printed circuit and a plurality of filled vias traversing some or all of these layers.

4. Probe according to claim 1, wherein the heat sink is porous and is least one of; open-cell pores, pores having a diameter within an interval of 10; 101 mm, pores of variable size, pores are one of increasing and decreasing size along a direction oriented towards a center of the cooling chamber.

5. Probe according to claim 1, wherein the heat transfer fluid is phase changing.

6. Probe according to claim 1, wherein the heat transfer fluid is a single-phase fluid, in the liquid state.

7. (canceled)

8. Probe according to claim 1, wherein the interface unit traverses the fluidtight wall and is arranged partly in the dry chamber and partly in the cooling chamber.

9. Probe according to claim 1, wherein a part of the interface unit is composed of at least one printed circuit located in the cooling chamber, said at least one printed circuit being mounted on both sides of the heat sink.

10. Probe according to claim 9, wherein part of the interface unit is composed of an intermediate board which traverses the fluidtight wall, the intermediate board being associated with said at least one printed circuit.

11. Probe according to claim 1, wherein at least part of the interface unit comprises a flexible or semi-rigid material.

12. Probe according to claim 1, wherein the interface unit is associated with the exterior of the probe by a plurality of cables, the plurality of cables being located in the dry chamber.

13-16. (canceled)

17. Probe according to claim 1, wherein the heat sink comprises several porous layers.

18. Probe according to claim 1, wherein the heat sink is in contact with the interface unit.

19. Probe according to claim 1, wherein the heat sink is arranged between said one or more at least one of emitting and receiving elements and the interface unit.

20. Probe according to claim 1, wherein the heat sink is obtained by powder sintering.

21. Probe according to claim 1, wherein the heat sink is a first heat sink, the probe comprising a second heat sink arranged one of in the dry chamber and on a face of the fluidtight wall in contact with the dry chamber.

22. Probe according to claim 21, wherein the second heat sink comprises a porous material.

23. Probe according to claim 1, wherein the heat sink at least one of has a shape complementary to the walls of the cooling chamber and at least partially fills the volume of the cooling chamber.

24. Probe according to claim 1, wherein the heat sink is in contact with at least one of both a rear face of said one or more at least one of emitting and receiving elements, and the fluidtight wall.

25. Probe according to claim 1, comprising at least one of a pressure sensor, an impact sensor, and a temperature sensor being one of within the interior and associated with the probe.

26. Probe according to claim 1, wherein said one or more at least one emitting and receiving elements for acoustic waves are arranged at a first end of the probe, the cooling chamber is arranged towards the first end of the probe, and the dry chamber is arranged towards a second end of the probe, the second end being opposite to the first end.
